(19) United States
(12) Patent Application Publication
(10) Pub. No.: US 2016/0082154 A1
(43) Pub. Date: Mar. 24, 2016

(54) Method and System for Treatment of Damaged Biological Tissue

(71) Applicant: CorMatrix Cardiovascular, Inc., Roswell, GA (US)

(72) Inventor: Robert G Matheny, Norcross, GA (US)

(21) Appl. No.: 14/960,348

(22) Filed: Dec. 5, 2015

Related U.S. Application Data
(63) Continuation-in-part of application No. 14/566,404, filed on Dec. 10, 2014.
Application No. 14/566,404 is a continuation-in-part of application No. 13/573,569, filed on Sep. 24, 2012, now Pat. No. 9,072,816.

(51) Int. Cl. A61L 27/36; A61L 27/54; A61L 27/58

(52) U.S. Cl. CPC A61L 27/3633; A61L 27/58; A61L 27/54; A61L 2430/20; A61L 2300/414; A61L 2300/406; A61L 2300/41; A61L 2300/42; A61L 2300/402; A61L 2300/408; A61L 2300/416; A61L 2300/434; A61L 2300/45

(57) ABSTRACT

Biomaterial compositions and articles comprising mesothelial tissue and a growth factor, for treating damaged biological tissue; particularly, damaged cardiovascular tissue. The biomaterial compositions and articles induce modulated healing, including modulation of inflammation and induced neovascularization, when administered to damaged biological tissue.

### CROSS-REFERENCES TO RELATED APPLICATIONS

This application is a continuation of U.S. application Ser. No. 14/566,404, filed on Dec. 10, 2014, which is a continuation-in-part of U.S. application Ser. No. 13/573,569, filed on Sep. 24, 2012, which is a continuation-in-part of U.S. application Ser. No. 11/334,631, filed on Jan. 18, 2006, now abandoned, which is a continuation of application Ser. No. 12/371,158, filed on Feb. 13, 2009, now abandoned, which is a continuation of application Ser. No. 11/747,018, filed on May 10, 2007, now abandoned.

### FIELD OF THE INVENTION

The present invention relates to methods for treating cardiovascular disorders. More particularly, the present invention relates to biomaterial compositions, articles and methods for treating damaged biological tissue; particularly, damaged cardiovascular tissue.

### BACKGROUND OF THE INVENTION

As is well known in the art, heart failure can be caused by a diverse array of cardiovascular disorders that reduce the efficiency of the myocardium, including ischemic heart disease, coronary artery disease, and a defective or diseased heart valve. Among the noted disorders, ischemic heart disease, which commonly presents as a myocardial infarction, is the leading cause of heart failure.

Indeed, in 2004 alone, the World Health Organization estimated that 12.2% of worldwide deaths occurred as a result of ischemic heart disease. Ischemic heart disease was also deemed the leading cause of death in middle to high income countries and second only to respiratory infections in lower income countries. The Global Burden of Disease: World Health Organization 2004 Update, Geneva (2008). Worldwide more than 3 million people present with a ST elevation myocardial infarction (STEMI) and 4 million people present with a non-ST elevation myocardial infarction (NSTEMI) a year. White, et al., Acute Myocardial Infarction, Lancet 372 (9638), pp. 570-84 (August 2008).

Rates of death from ischemic heart disease have slowed or declined in most high income countries, although cardiovascular disease still accounted for 1 in 3 of all deaths in the USA in 2008. Roger, et al., Executive summary: Heart Disease and Stroke Statistics—2012 update: A report from the American Heart Association, Circulation 125 (1), pp. 188-97 (January 2012).

In contrast, ischemic heart disease is becoming a more common cause of death in the developing world. For example in India, ischemic heart disease had become the leading cause of death by 2004; accounting for 1.46 million deaths (14% of total deaths). Deaths in India due to ischemic heart disease were also expected to double during 1985-2015. Gupta, et al., Epidemiology and Causation of Coronary Heart Disease and Stroke in India, Heart 94 (1), pp. 16-26 (January 2008).

Globally, it is predicted that disability adjusted life years (DALYs) lost to ischemic heart disease will account for 5.5% of total DALYs in 2030, making it the second most important cause of disability (after unipolar depressive disorder), as well as the leading cause of death by this date.

Ischemic heart disease often occurs when myocardial tissue is no longer receiving adequate blood flow. Various methods for treating ischemic heart disease have thus been developed. Such methods include systemic delivery of various pharmacological agents.

Several additional methods for treating ischemic heart disease are directed to re-establishing blood flow to the ischemic area. Such methods include stimulation of angiogenesis and surgical intervention, e.g. bypass surgery or angioplasty. Other methods include the use of lasers to bore holes through the ischemic area(s) to promote blood flow. As one can readily appreciate, there are numerous incumbent risks associated with the noted methods.

A further method for treating ischemic heart disease is the direct delivery of bioactive or pharmacological agents to the ischemic area. Illustrative is the delivery of extracellular matrix (ECM) based compositions directly to cardiovascular tissue disclosed in Co-pending application Ser. No. 13/573,569.

More recently, ventricular assist devices (VADs) have been employed as treatment platforms for various pharmacological therapies, e.g. stem cell administration. VADs are designed to support (or augment) the function of either the right (RVAD) or left (LVAD) ventricle, or both at once (BiVAD). The type of VAD employed depends primarily on the underlying cardiovascular disorder, and the pulmonary arterial resistance that determines the load on the right ventricle.

Although the direct delivery of bioactive or pharmacological agents; particularly, the ECM based compositions disclosed in Co-pending application Ser. No. 13/573,569, and other treatment therapies employing ventricular assistance have been found effective to treat cardiovascular disorders and, thereby, heart failure, there remains a need to provide even more effective means for treating cardiovascular disorders.

It is therefore an object of the present invention to provide improved compositions, articles and methods for treating damaged cardiovascular tissue and, thereby, cardiovascular disorders.

It is another object of the present invention to provide biomaterial compositions, articles and methods for treating damaged cardiovascular tissue and, thereby, cardiovascular disorders, which, when delivered to damaged biological tissue; particularly, cardiovascular tissue, modulates inflammation of the damaged tissue and induces neovascularization, tissue proliferation, bioremodeling, and regeneration of cardiovascular tissue and associated structures with site-specific structural and functional properties.

### SUMMARY OF THE INVENTION

The present invention is directed to biomaterial compositions, articles and methods for treating damaged biological tissue; particularly, damaged cardiovascular tissue.

In some embodiments of the invention, the biomaterial compositions comprise an extracellular matrix (ECM) composition comprising at least one ECM material.

In some embodiments of the invention, the biomaterial articles comprise comprise a particulate structure or component comprising an ECM composition.

In some embodiments of the invention, the ECM composition is encased in an ECM-mimicking biomaterial composition.

In a preferred embodiment, the ECM material is derived from a mammalian tissue source selected from the group comprising small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, omentum extracellular matrix, cardiac extracellular matrix, e.g., pericardium and/or myocardium, kidney extracellular matrix, pancreas extracellular matrix, lung extracellular matrix, and combinations thereof.

In some embodiments, the mammalian tissue source comprises the basement membrane of mammalian tissue/organs selected from the group comprising urinary basement membrane (UBM), liver basement membrane (LBM), and amnion, chorion, allograft pericardium, allograft acellular dermis, amniotic membrane, Wharton's jelly, and combinations thereof.

In a preferred embodiment, the ECM materials comprise decellularized (or acellular) ECM.

In some embodiments, the biomaterial compositions and/or articles further comprise an exogenously added biologically active agent.

In some embodiments, the biologically active agent comprises a growth factor selected from the group consisting of transforming growth factor alpha (TGF-α), transforming growth factor beta (TGF-β), fibroblast growth factor-2 (FGF-2), basic fibroblast growth factor (bFGF), vascular epithelial growth factor (VEGF), and insulin-like growth factor (IGF).

In some embodiments, the biologically active agent comprises a cell selected from the group consisting of an embryonic stem cell, mesenchymal stem cell, hematopoietic stem cell, bone marrow stem cell, bone marrow-derived progenitor cell, myosatellite progenitor cell, totipotent stem cell, pluripotent stem cell, multipotent stem cell, oligopotent stem cell and unipotent stem cell.

In some embodiments, the biologically active agent comprises a protein selected from the group consisting of collagen (types I-V), proteoglycans, glycosaminoglycans (GAGs), glycoproteins, cytokines, cell-surface associated proteins, and cell adhesion molecules (CAMs).

In some embodiments, the biologically active agent comprises statin selected from the group consisting of atorvastatin, cerivastatin, fluvastatin, lovastatin, mevastatin, pitavastatin, pravastatin, rosuvastatin and simvastatin.

In some embodiments, the biomaterial compositions and/or articles further comprise a pharmacological agent.

In some embodiments, the pharmacological agent comprises an agent selected from the group consisting of an anti-viral agent, analgesic, antibiotic, anti-inflammatory, anti-neoplastic, anti-spasmodic, enzyme and enzyme inhibitor, anticoagulant and/or antithrombic agent, and vasodilating agent.

In a preferred embodiment of the invention, the biomaterial compositions and articles of the invention are configured (or formulated) to induce “modulated healing”, as defined herein, when delivered to the damaged tissue.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a front sectional view of one embodiment of a biomaterial particulate article of the invention;

FIG. 2 is a depiction of a normal mammalian heart; and

FIG. 3 is a depiction of a mammalian heart having an ischemic infracted region.

### DETAILED DESCRIPTION OF THE PREFERRED EMBODIMENT

Before describing the present invention in detail, it is to be understood that this invention is not limited to particularly exemplified apparatus, systems, compositions or methods as such may, of course, vary. Thus, although a number of systems, compositions and methods similar or equivalent to those described herein can be used in the practice of the present invention, the preferred systems, compositions and methods are described herein.

It is also to be understood that, although a preferred method of delivering an ECM particulate article and, hence, composition formed therefrom to biological tissue comprises direct injection into the tissue. The delivery of an ECM particulate article and composition formed therefrom is not limited to direct injection. According to the invention, an ECM particulate article and composition formed therefrom of the invention can be delivered to biological tissue by other conventional means, including topical administration.

It is further to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an anti-inflammatory” includes two or more such agents and the like. Definitions

The term “particulate”, as used herein, means and includes a particulate article or structure having a mean particle size in the range of 20-2000 microns.

The terms “cardiovascular disorder” and “heart failure” are used interchangeably herein, and mean and include any abnormal function of the heart; particularly, abnormal functions or deficiency of the myocardium. The terms “cardiovascular disorder” and “heart failure” thus include, without limitation, ischemic heart disease, coronary artery disease, a defective or diseased heart valve, myocarditis, an inflammatory disease, cardiomyopathy and amyloidosis.

The terms “cardiovascular tissue damage,” “cardiac tissue damage,” and “cardiac tissue injury” and are used interchangeably herein, and mean and include any area of abnormal tissue in the cardiovascular system or heart caused by a disease, disorder, injury or damage, including damage to the epicardium, endocardium and/or myocardium.

As is well known in the art, cardiovascular tissue damage most often involves damage or injury to the myocardium and, therefore, for the purposes of this disclosure, myocardial damage or injury is equivalent to cardiovascular tissue damage.

The term “chamber remodeling”, as used herein, means and includes a series of events (which may include changes in gene expression, molecular, cellular and interstitial changes) that result in changes in size, shape and function of biological tissue following stress or injury. As is well known in the art, remodeling can occur after a myocardial infarction, pressure overload (e.g., aortic stenosis, hypertension), volume overload (e.g., valvular regurgitation), inflammatory heart disease (e.g., myocarditis), or in idiopathic cases (e.g., idiopathic dilated cardiomyopathy).

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The terms “extracellular matrix”, “ECM” and “ECM material” are used interchangeably herein, and mean and include a collagen-rich substance that is found in between cells in mammalian tissue, and any material processed therefrom, e.g. decellularized ECM. According to the invention, the ECM material can be derived from a variety of mammalian tissue sources, including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, omomentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

According to the invention, ECM material can comprise, in whole or in part, just the basement membrane (or transitional epithelial layer) with the subadjacent tunica propria, the tunica submucosa, tunica muscularis, and tunica serosa. The extracellular matrix component of the ECM material can thus contain any or all of these layers or only the basement membrane portion, excluding the submucosa.

In a preferred embodiment, the ECM material comprises decellularized (or acellular) ECM.

The terms “ECM-mimicking biomaterial” and “ECM-mimicking material” are used interchangeably herein, and mean and include a biodegradable biomaterial that induces neovascularization and bioremodeling of tissue in vivo, i.e. when disposed proximate damaged biological tissue. The term “ECM-mimicking material” thus includes, without limitation, ECM-mimicking polymeric biomaterial s; specifically, poly(glycerol sebacate) (PGS).

The term “biologically active agent”, as used herein, means and includes an agent that induces or modulates a physiological or biological process, or cellular activity, e.g., induces proliferation, and/or growth and/or regeneration of tissue.

The term “biologically active agent” thus means and includes, without limitation, the following growth factors: platelet derived growth factor (PDGF), epidermal growth factor (EGF), transforming growth factor alpha (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor alpha (TNF-α), and placental growth factor (PLGF).

The term “biologically active agent” also means and includes, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells. The group also comprises cardiomyocytes, myoblasts, monocytes, parenchymal cells, epithelial cells, endothelial cells, mesothelial cells, fibroblasts, osteoblasts, chondrocytes, exogenous cells, endogenous cells, macrophages, capillary endothelial cells, autologous cells, xenogenic cells, allogenic cells, and cells derived from any of the three germ layers including the endoderm, mesoderm and ectoderm.

The term “biologically active agent” also means and includes, without limitation, the following biologically active agents (referred to interchangeably herein as a “protein”, “peptide” and “polypeptide”): collagen (types I-V), proteoglycans, glycosaminoglycans (GAGs), glycoproteins, cytokines, cell-surface associated proteins, cell adhesion molecules (CAM), endothelial ligands, matrikines, cadherins, immuoglobins, fibril collagens, non-fibrillar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, fibulin II, integrins, transmembrane molecules, thrombospondins, ostepontins, and angiotensin converting enzymes (ACE).

The term “biologically active composition”, as used herein, means and includes a composition comprising at least one “biologically active agent.”

The term “pharmacological agent”, as used herein, means and includes an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “pharmacological agent” thus means and includes, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The term “pharmacological agent” thus includes, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti-VEGFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The term “pharmacological agent” further means and includes the following Class I-Class V anti-arrhythmic agents: (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem and (Class V) adenosine and digoxin.

The term “pharmacological agent” further means and includes, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

As indicated above, the term “pharmacological agent” further means and includes, without limitation, an anti-inflammatory.

The term “anti-inflammatory”, as used herein, means and includes an agent that prevents or treats biological tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “pharmacological composition”, as used herein, means and includes a composition comprising a “pharmacological agent”.

The terms “prevent” and “preventing” are used interchangeably herein, and mean and include reducing the frequency or severity of a disease, condition or disorder. The term does not require an absolute preclusion of the disease, condition or disorder. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition or disorder. The terms include “active treatment”, i.e. treatment directed specifically toward the improvement of a disease, pathological condition or disorder, and “causal treatment”, i.e. treatment directed toward removal of the cause of the associated disease, pathological condition or disorder.

The terms “treat” and “treatment” further include “palliative treatment”, i.e. treatment designed for the relief of symptoms rather than the curing of the disease, pathological condition or disorder, “preventative treatment”, i.e. treatment directed to minimizing or partially or completely inhibiting the development of the associated disease, pathological condition or disorder, and “supportive treatment”, i.e. treatment employed to supplement another specific therapy directed toward the improvement of the associated disease, pathological condition or disorder.

The term “therapeutically effective”, as used herein, means that the amount of an “biologically active agent”, “biologically active composition”, “pharmacological agent” and/or “pharmacological composition” administered to a mammalian organ or biological tissue is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “delivery” and “administration” are used interchangeably herein, and mean and include providing a “pharmacological composition” or “biologically active agent” or “active agent formulation” to a treatment site, e.g., damaged tissue, through any method appropriate to deliver the functional agent or formulation or composition to the treatment site. Non-limiting examples of delivery methods include direct injection, percutaneous delivery and topical application at the treatment site.

The term “percutaneous”, as used herein, means and includes any penetration through the skin of a patient or subject, whether in the form of a small cut, incision, hole, cannula, tubular access sleeve or port or the like.

The terms “patient” and “subject” are used interchangeably herein, and mean and include warn blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As indicated above, the present invention is directed to biomaterial compositions, articles and methods for treating damaged biological tissue; particularly, damaged cardiovascular tissue.

In some embodiments of the invention, the biomaterial compositions comprise an extracellular matrix (ECM) composition comprising at least one ECM material.

In some embodiments of the invention, the biomaterial articles comprise comprise a particulate structure or component comprising an ECM composition.

In some embodiments of the invention, the ECM composition is encased in an ECM-mimicking biomaterial composition.

In a preferred embodiment, the ECM-mimicking biomaterial composition comprises poly(glycerol sebacate) (PGS).

As set forth in Co-Pending application Ser. No. 14/554,730, PGS exhibits numerous beneficial properties that provide several beneficial biochemical actions or activities; particularly, ECM-mimicking properties and actions.

Indeed, it has been found that PGS induces tissue remodeling and regeneration when administered proximate to damaged tissue, thus, mimicking the seminal regenerative properties of ECM and, hence, an ECM composition formed therefrom. The mechanism underlying this behavior is deemed to be based on the mechanical and biodegradation kinetics of the PGS. See Sant, et al., Effect of Biodegradation and de novo Matrix Synthesis on the Mechanical Properties of VIC-seeded PGS-PCL scaffolds, Acta. Biomater., vol. 9(4), pp. 5963-73 (2013).

As indicated above, in a preferred embodiment, the ECM material employed in the biomaterial compositions and articles of the invention is derived from a mammalian tissue source selected from the group comprising small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornomentum extracellular matrix, cardiac extracellular matrix, e.g., pericardium and/or myocardium, kidney extracellular matrix, pancreas extracellular matrix, lung extracellular matrix, and combinations thereof.

According to the invention, the ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

According to the invention, the biomaterial compositions of the invention can also comprise ECM material from two or more mammalian sources. Thus, for example, the composition can comprise ECM material combinations from such sources as, for example, but not limited to, SIS, LBM, SS, UBS, placental basement membrane, pancreatic basement membrane, large intestine submucosa, lung interstitial membrane, respiratory tract submucosa, heart ECM, dermal matrix, and, in general, ECM material from any mammalian fetal tissue. The ECM material sources can also comprise different mammalian animals or an entirely different species of mammals.

The ECM material can also be used in whole or in part, so that, for example, an ECM material can contain just the basement membrane (or transitional epithelial layer) with the subadjacent tunica propria, the tunica submucosa, tunica muscularis, and tunica serosa. The ECM material component of the composition can contain any or all of these layers, and thus could conceivably contain only the basement membrane portion, excluding the submucosa. However, generally, and especially since the submucosa is thought to contain and support the active growth factors and other proteins necessary for in vivo tissue regeneration, the ECM or matrix composition from any given source will contain the active extracellular matrix portions that support cell development and differentiation and tissue regeneration.

As indicated above, in a preferred embodiment of the invention, the ECM material comprises acellular ECM.

According to the invention, the ECM can also be sterilized via applicant's proprietary novasterilis process disclosed in Co-Pending U.S. application Ser. No. 13/480,205; which is expressly incorporated by reference herein in its entirety.

According to the invention, the ECM material can be formed into a particulate to form particulate ECM components of the invention and fluidized, as described in U.S. Pat. Nos. 5,275,826, 6,579,538 and 6,933,326, to form an ECM composition of the invention.

According to the invention, various conventional means can be employed to form a particulate ECM material and, hence, component. In some embodiments, the ECM material is formed into a sheet, fluidized (or hydrated), if necessary, frozen and ground.

In some embodiments of the invention, the ground ECM material is subsequently filtered to achieve a desired particulate size. Thus, in some embodiments, the ECM material and, hence, component has a particulate size no greater than 2000 microns. In some embodiments, the ECM material and, hence, component preferably has a particulate size no greater than 500 microns. In a preferred embodiment, the ECM material and, hence, component has a particulate size in the range of about 20 microns to about 300 microns.

Thus, in some embodiments of the invention, the biomaterial articles comprise a particulate component comprising an ECM composition comprising at least one ECM material.

As indicated above, in some embodiments of the invention, the biomaterial compositions and/or articles further comprise at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents, including, without limitation, the aforementioned cells, proteins and growth factors.

In some embodiments of the invention, the biologically active agent comprises a protein selected from the group comprising, without limitation, collagen (types I-V), proteoglycans, glycosaminoglycans (GAGs), glycoproteins, cytokines, cell-surface associated proteins, and cell adhesion molecules (CAMs).

In some embodiments, the biologically active agent provides a structural support scaffold. Suitable bioactive agents include, without limitation, elastin and ECM having additional GAG content, such as additional hyaluronic acid and/or chondroitin sulfate.

In some embodiments, the biomaterial compositions and/or articles further comprise at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

According to the invention, the amount of a pharmacological agent added to a biomaterial composition of the invention will, of course, vary from agent to agent. For example, in one embodiment, wherein the pharmacological agent comprises dicloflenac (Voltaren®), the amount of dicloflenac included in the biomaterial composition is preferably in the range of 10 μg-75 mg.

In some embodiments of the invention, the pharmacological agent specifically comprises an anti-inflammatory agent. According to the invention, suitable anti-inflammatory agents include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

According to the invention, the amount of an anti-inflammatory added to a biomaterial composition and/or article of the invention can similarly vary from anti-inflammatory to anti-inflammatory. For example, in one embodiment of the invention, wherein the pharmacological agent comprises ibuprofen (Advil®), the amount of ibuprofen included in the biomaterial composition is preferably in the range of 100 μg-200 mg.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. In particular, Applicant has found that when a statin is added to ECM (wherein a statin augmented ECM composition is formed) and the statin augmented ECM composition is administered to damaged tissue, the statin interacts with the cells recruited by the ECM, wherein the statin augmented ECM composition modulates inflammation of the damaged tissue by modulating several significant inflammatory processes, including restricting expression of monocyte chemoattractant protein-1 (MCP-1) and chemokine (C-C) motif ligand 2 (CCR2).

Further beneficial biochemical actions are discussed in detail in Applicant's Co-Pending application Ser. No. 13/328,287, filed on Dec. 16, 2011, Ser. No. 13/373,569, filed on Sep. 24, 2012 and Ser. No. 13/782,024, filed on Mar. 1, 2013; which are incorporated by reference herein in their entirety.

According to the invention, the amount of a statin added to a biomaterial composition and/or article of the invention is preferably less than 20 mg, more preferably, less than approximately 10 mg.

Additional suitable pharmacological agents and compositions that can be delivered within the scope of the invention are disclosed in Pat. Pub. Nos. 20070014874, 20070014873, 20070014872, 20070014871, 20070014870, 20070014869, and 20070014868; which are expressly incorporated by reference herein in its entirety.

A cording to the invention, the aforementioned biologically active and pharmacological agents can be incorporated into any of the ECM and ECM-mimicking compositions of the invention.

According to the invention, the ECM materials and, hence, biomaterial compositions formed therefrom can comprise mixed liquids, mixed emulsions, mixed gels, mixed pastes, or mixed solid particulates, e.g. particulate ECM components. The liquid or semi-solid components of the biomaterial compositions can also comprise various concentrations.

Preferably, the concentration of the liquid or semi-solid components of the biomaterial compositions is in the range of about 0.001 mg/ml to about 200 mg/ml. Suitable concentration ranges thus include, without limitation: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml and about 40 mg/ml. to about 42 mg/ml.

The noted concentration ranges are, however, merely exemplary and not intended to be exhaustive or limiting. It is understood that any value within any of the listed ranges is deemed a reasonable and useful value for a concentration of a liquid or semi-solid component of a biomaterial composition of the invention.

As indicated above, in some embodiments of the invention, the biomaterial compositions are formulated to be injected into damaged or cardiovascular tissue, i.e. particulate biomaterial compositions. According to the invention, the particulate biomaterial compositions can thus comprise various desired proportions of particulate, e.g. particulate ECM components, and fluidizing or hydrolyzing material. By way of example, in some embodiments, the biomaterial compositions comprise approximately 90% particulate ECM components and approximately 10% saline.

As also indicated above, the biomaterial compositions of the invention can also be formulated into glue compositions.

According to the invention, the biologically active and pharmacological agents referenced above can also comprise various forms. In some embodiments of the invention, the biologically active and pharmacological agents, e.g. simvastatin, comprise microcapsules that provide delayed delivery of the agent contained therein.

According to the invention, upon delivery of a biomaterial composition and/or article to damaged biological tissue, “modulated healing” is effectuated.

The term “modulated healing”, as used herein, and variants of this language mean and include modulation (e.g., alteration, delay, retardation, reduction, etc.) of a process involving different cascades or sequences of naturally occurring tissue repair in response to localized tissue damage or injury, substantially reducing their inflammatory effect. Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

For example, in some embodiments, the biomaterial compositions and/or articles are specifically configured (or formulated) to alter, delay, retard, reduce, and/or detain one or more of the phases associated with healing of damaged tissue, including, but not limited to, the inflammatory phase (e.g., platelet or fibrin deposition), and the proliferative phase when in contact with biological tissue.

In some embodiments of the invention, “modulated healing” means and includes the ability of a biomaterial composition and/or article to restrict the expression of inflammatory components. By way of example, according to the invention, when a biomaterial composition and/or article comprising ECM and a statin is delivered to or disposed proximate damaged biological tissue, the biomaterial composition restricts expression of monocyte chemoattractant protein-1 (MCP-1) and chemokine (C-C) motif ligand 2 (CCR2).

In some embodiments, “modulated healing” means and includes the ability of a biomaterial composition and/or article to alter a substantial inflammatory phase (e.g., platelet or fibrin deposition) at the beginning of the tissue healing process. As used herein, the phrase “alter a substantial inflammatory phase” refers to the ability of a biomaterial composition and/or article to substantially reduce the inflammatory response at an injury site when in contact with biological tissue.

In such an instance, a minor amount of inflammation may ensue in response to tissue injury, but this level of inflammation response, e.g., platelet and/or fibrin deposition, is substantially reduced when compared to inflammation that takes place in the absence of a biomaterial composition and/or article of the invention.

The term “modulated healing” also refers to the ability of a biomaterial composition and/or article to induce host tissue proliferation, bioremodeling, including neovascularization, e.g., vasculogenesis, angiogenesis, and intussusception, and regeneration of tissue structures with site-specific structural and functional properties.

Thus, in some embodiments, the term “modulated healing” means and includes the ability of a biomaterial composition and/or article to modulate inflammation and/or induce host tissue proliferation and remodeling. Again, by way of example, according to the invention, when a when a biomaterial composition (and/or article) comprising ECM and a statin is delivered to or disposed proximate damaged biological tissue, the stain interacts with cells recruited by the ECM, wherein the biomaterial composition and/or article modulates inflammation by, among other actions, restricting expression of monocyte chemoattractant protein-1 (MCP-1) and chemokine (C-C) motif ligand 2 (CCR2), and induces tissue proliferation, bioremodeling and regeneration of tissue structures with site-specific structural and functional properties.

By way of a further example, according to the invention, when a biomaterial composition and/or article comprising ECM and an exogenously added growth factor, e.g. TGF-β, is disposed proximate damaged biological tissue, the growth factor similarly interacts with the ECM and cells recruited by the ECM, wherein the biomaterial composition and/or article similarly modulates inflammation and induces tissue proliferation, bioremodeling and regeneration of tissue.

In some embodiments of the invention, the biomaterial compositions and/or articles comprise a single-stage delivery vehicle, wherein a modulated degradation rate or dosage of a biomaterial composition or article and/or biologically active and/or pharmacological agent of the invention is provided.

According to the invention, the term “modulated dosage” as used herein, and variants of this language generally refer to the modulation or control (e.g., alteration, delay, retardation, reduction, etc.) of the delivery rate of an ECM composition, and/or biologically active and/or pharmacological agent of the invention into biological tissue.

The term “modulated degradation rate” as used herein, and variants of this language generally refer to the modulation or control of the degradation or dispersal rate of biomaterial composition and/or article of the invention within biological tissue.

In some embodiments of the invention, the biomaterial compositions and/or articles comprise a multi-stage agent delivery profile, wherein a plurality of the aforementioned biologically active and/or pharmacological agents is administered via a modulated dosage. In some embodiments, the multi-stage delivery vehicle can thus comprise a combination of different ECM compositions and/or different biologically active and/or pharmacological agents. By way of example, in some embodiments, the multi-stage delivery vehicle comprises a biomaterial article comprising a particulate component comprising an ECM composition comprising a growth factor that is encased in an ECM-mimicking composition comprising an anti-inflammatory.

In some embodiments of the invention, the biomaterial compositions and/or articles are configured to change consistency in response to a physiological condition. Thus, the biomaterial compositions and/or articles can be a certain consistency outside the body, e.g. a solid, and when placed in the body the consistency can change, e.g. a gel, in response to the change in pH, temperature, or enzymatic activity present in the body at the site of placement of the compositions and/or articles.

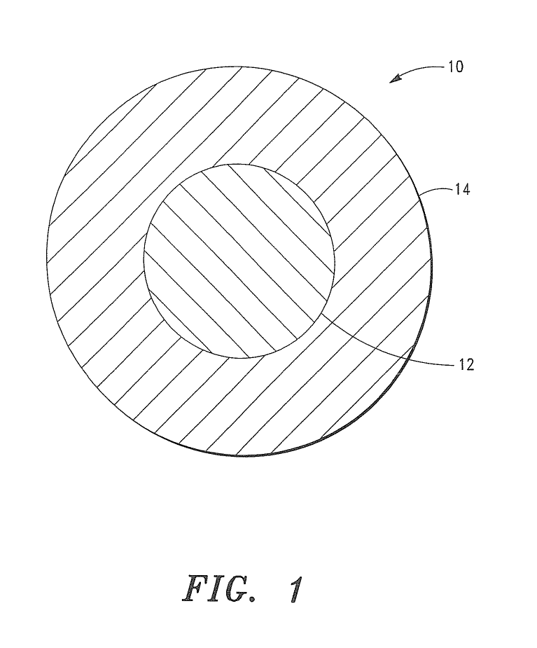

Referring now to FIG. 1, there is shown one embodiment of a biomaterial article of the invention. As illustrated in FIG. 1, the biomaterial article 10 comprises a particulate article comprising a particulate component 12 comprising a first composition that is encased in a second composition 14.

As indicated above, according to the invention, the first composition of the particulate component 12 can comprise an ECM composition and the second composition 14 can comprise an ECM-mimicking composition.

In a preferred embodiment of the invention, a plurality of biomaterial articles shown in FIG. 1 are employed to form a particulate composition for treating damaged tissue.

Thus, in some embodiments, the particulate composition comprises a plurality of particulate components comprising an ECM composition, the ECM composition comprising at least one ECM material, the particulate composition being configured to induce modulated healing when delivered to damaged biological tissue.

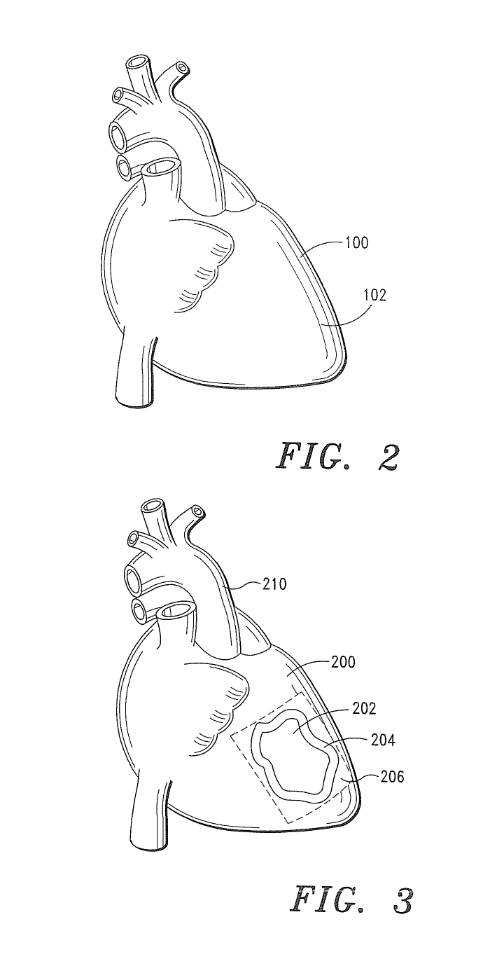

Referring now to FIG. 2, there is shown a depiction of a normal human heart 100. The heart wall 102 consists of an inner layer of simple squamous epithelium, referred to as the endocardium. The endocardium overlays the myocardium (a variably thick heart muscle) and is enveloped within a multi-layer tissue structure referred to as the pericardium. The innermost layer of the pericardium, referred to as the visceral pericardium or epicardium, covers the myocardium. An outermost layer of the pericardium, referred to as the fibrous pericardium, attaches the parietal pericardium to the sternum, the great vessels and the diaphragm.

Referring now to FIG. 3, there is shown a depiction of a heart 200 having an ischemic infarcted region 202, and a peri-infarcted region 204 that is surrounded by healthy non-ischemic myocardium tissue 206.

As indicated above, the ischemic infarcted region 202 (or myocardial infarction) can, and in many instances will trigger a cascading sequence of myocellular events. In many instances, the myocellular events lead to deterioration in ventricular function and heart failure.

According to the invention, the effects of an ischemic infarcted region, such as infarct region 202, can be ameliorated or eliminated by delivering a biomaterial composition and/or article (and/or a plurality thereof) of the invention directly to the infarcted cardiovascular tissue. As stated above, the biomaterial composition and/or article(s) will induce modulated healing of the damaged tissue (e.g., infarct region 202), including modulating inflammation of the damaged tissue and inducing neovascularization, tissue proliferation, bioremodeling, and regeneration of new cardiac tissue structures with site-specific structural and functional properties.

According to the invention, the biomaterial compositions and/or article(s) can be delivered to infarcted cardiovascular tissue, such as tissue 202, as well as other damaged or diseased biological tissue, by various conventional means. In some embodiments, a multi-needle injection system, such as disclosed in U.S. application Ser. No. 13/782,115, filed Sep. 19, 2012 is employed to deliver one or more biomaterial compositions to damaged or diseased cardiovascular tissue.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A composition for treating damaged biological tissue, Comprising:
an extracellular matrix (ECM) composition comprising acellular mesothelial tissue and a supplemental biologically active agent, said supplemental biologically active agent comprising a growth factor selected from the group consisting of transforming growth factor beta (TGF-beta), fibroblast growth factor-2 (FGF-2) and vascular epithelial growth factor (VEGF), wherein, when said ECM composition is administered to said damaged biological tissue, said ECM composition induces modulated healing, said modulated healing comprising modulation of inflammation and induced bioremodeling and regeneration of new biological tissue.

2. The composition of claim 1, wherein said ECM composition further comprises a statin selected from the group consisting of atorvastatin, cerivastatin, fluvastatin, lovastatin, mevastatin, pitavastatin, pravastatin, rosuvastatin and simvastatin.

3. The composition of claim 1, wherein said ECM composition further comprises a pharmacological agent.

4. The composition of claim 4, wherein said pharmacological agent comprises an agent selected from the group consisting of an anti-viral agent, analgesic, antibiotic, anti-inflammatory, anti-neoplastic, anti-spasmodic, enzyme and enzyme inhibitor, anticoagulant and/or antithrombic agent, and vasodilating agent.
